(19) United States
(12) Patent Application Publication
(10) Pub. No.: US 2024/0052265 A1
(43) Pub. Date: Feb. 15, 2024

(54) COMPOSITION TO PREVENT OR REDUCE SPLASHING ON USE OF A TOILET

(71) Applicant: Loren Azouri, Tel Aviv (IL)

(72) Inventor: Loren Azouri, Tel Aviv (IL)

(21) Appl. No.: 18/266,819

(22) PCT Filed: Oct. 31, 2022
(86) PCT No.: PCT/IL2022/051154

§ 371 Date: Jun. 13, 2023

Related U.S. Application Data
(60) Provisional application No. 63/280,161, filed on Nov. 17, 2021.

(51) Int. Cl. C11D 11/00; C11D 3/04; C11D 3/10; C11D 1/00

(52) U.S. Cl. CPC C11D 11/0058; C11D 3/042; C11D 3/10; C11D 1/002

(57) ABSTRACT

A composition for use in reducing splashing on excretion into a toilet, and optionally, the composition may be used to reduce noise and/or odor and/or to disinfect and/or to prevent accumulation of dirt on the walls of the toilet.

### RELATED APPLICATION/S

This application claims the benefit of priority under 35 USC § 119(e) of U.S. Provisional Patent Application No. 63/280,161 filed 17 Nov. 2021, the contents of which are incorporated herein by reference in their entirety.

### BACKGROUND OF THE INVENTION

The current invention, in some embodiments thereof, relates to a composition for use in reducing splashing on excretion into a toilet.

WO 2021/123511, WO 2016/017829 and CN 104559921 disclose foaming compositions containing an acid and a base, and a surfactant, wherein the base and the acid react in the presence of water, producing carbon dioxide, to produce a foam that acts as a cushion and allows excrement to be hidden, and splashes, odors, and noises to be avoided.

Additional devices for producing foams are attached to the toilet or urinal walls, as disclosed in U.S. Pat. No. 5,958,334 and KR 101025040 B1, planar sheets inserted into the toilet bowl as disclosed by US 2007/0039089, or floating tablets as disclosed by DE 10301097 and JPH 0913466.

### SUMMARY OF THE INVENTION

According to an aspect of some embodiments of the invention, there is provided a composition to reduce or prevent splashing on use of a toilet including: 1/10-30% wt/wt of one or more surfactants; 5-40% wt/wt of one or more carbon dioxide evolving compounds; and 5-40% wt/wt of one or more acids.

According to some embodiments of the invention, the composition further includes a disintegrant.

According to some embodiments of the invention, the composition further includes one or more additional excipients selected from the group consisting of an odorant, a deodorant, a thickener, a disintegrant, a binder, a moisture absorbing agent, a disinfectant, a cleaning agent, a foam promoting agent, a colorant, and combinations thereof.

According to some embodiments of the invention, at least one the additional excipients in the composition serves a dual purpose.

According to some embodiments of the invention, the one or more surfactant includes at least one of anionic surfactants, cationic surfactants, amphoteric surfactants, non-ionic surfactants, saponins, proteins, or a combination thereof.

According to some embodiments of the invention, the one or more surfactants includes a natural or synthetic compound.

According to some embodiments of the invention, the one or more surfactants includes at least one compound selected from the group consisting of alkylnaphthalene sulfonates, fatty alcohol sulphates or sulphonates which may be unsubstituted or substituted salts, adducts and derivatives thereof.

According to some embodiments of the invention, the one or more surfactants includes at least one compound selected from the group selected from the group consisting of an ammonium salt, a sodium salt, ammonium lauryl sulfate, sodium laureth sulfate, sodium lauryl sarcosinate, sodium myreth sulfate, sodium pareth sulfate, sodium stearte, sodium lauryl sulfate, α-olefin sulfonate, ammonium laureth sulfate, sodium isopropyl naphthalene sulfonate, sodium polystyrene sulfonate, and salts, adducts, derivatives, and combinations thereof.

According to some embodiments of the invention, the one or more surfactants includes at least one compound selected from the group consisting of quaternary ammonium salts in the form of halides, halogenated lower alkyl or benzyl or hydroxy-lower alkyl radicals, alkylsulfates which may be substituted or unsubstituted by halogens, alkyl group, and or aryl groups, and/or salts, adducts adducts, derivatives, and combinations thereof.

According to some embodiments of the invention, the one or more surfactants includes at least one compound selected from the group consisting of aminocarboxylic acids and aminosulphonic acids, alkyl and alkylamido betaines, higher fatty alkanolamides, salts, adducts adducts, derivatives and combinations thereof.

According to some embodiments of the invention, t the one or more surfactants includes at least one compound selected from the group consisting of polyalkylglycol ether or ester derivatives of aliphatic, cycloaliphatic or aryl alcohols, saturated or unsaturated fatty acids and alkylphenols, water-soluble glycol adducts of polyethylene oxide, fatty acid esters of polyoxyethylene sorbitan, salts, adducts, derivatives, and combinations thereof.

According to some embodiments of the invention, the one or more carbon dioxide evolving compound includes a carbonate.

According to some embodiments of the invention, the one or more carbon dioxide evolving compound is selected from ammonium carbonate, sodium carbonate, potassium carbonate, ammonium bicarbonate, sodium bicarbonate, potassium bicarbonate, adducts, derivatives or combinations thereof.

According to some embodiments of the invention, the at least one acid is organic or inorganic.

According to some embodiments of the invention, the at least one the acid is selected from the group consisting of citric acid, tartaric acid, oleic acid, stearic acid, palmitic acid, glucuronic acid, α-hydroxybutyric acid, α-hydroxyisobutyric acid, mandelic acid, pyruvic acid, malic acid, citramalic acid, mucic acid, glycolic acid, hydroxyglutaric acid, lactic acid, catechol acid, oxalic acid, succinic acid, tartaric acid, salicyclic acid, adipic acid, ascorbic acid, acetic acid, boric acid, hydrochloric acid, hydrobromic acid, sulfuric acid, sulphamic acid, nitric acid, and any combination thereof.

According to some embodiments of the invention, the at least one disintegrant is selected from the group consisting of starch, partially pregelatinized starch, chitosan, mucilage, croscarmellose sodium, crospovidone, sodium starch glycolate, microcrystalline cellulose, and combinations thereof.

According to some embodiments of the invention, the additional excipient is a moisture absorbing agent.

According to some embodiments of the invention, the moisture absorbing agent is included in a coating.

According to some embodiments of the invention, the moisture absorbing agent is selected from the group consisting of aluminum oxide, calcium chloride, calcium sulfate, activated carbon, zeolites, silica, and a combination thereof.

According to some embodiments of the invention, the additional excipient is an odorant.

According to some embodiments of the invention, the odorant is a perfume or fragrance.

According to some embodiments of the invention, the additional excipient is a deodorant.

According to some embodiments of the invention, the deodorant is a methane gas or hydrogen sulfide neutralizer.

According to some embodiments of the invention, the additional excipient is a binder.

According to some embodiments of the invention, the binder is selected from the group consisting of starch, pregelatinized starch, Sodium alginate, gelatin, polyvinyl pyrrolidone (PVP), methylcellulose, hydroxy propyl methyl cellulose (HPMC), polymethacrylates, sodium carboxy methyl cellulose, polyethylene glycol (PEG), methylcellulose, cellulose derivatives, polyvinylpyrrolidone, microcrystalline cellulose, and combinations thereof.

According to some embodiments of the invention, the additional excipient is a disinfectant.

According to some embodiments of the invention, the disinfectant is selected from peroxides, percarbonates, peracids, chlorine compounds, phenolics, quaternary ammonium compounds, or combinations thereof.

According to some embodiments of the invention, the additional excipient is a cleaning agent.

According to some embodiments of the invention, the additional excipient is a foam promoting agent.

According to some embodiments of the invention, the foam promoting agent is a buffer, a frothing agent, a foam stabilizer, a thickening agent, a foam booster, or combinations thereof.

According to some embodiments of the invention, the composition further includes a water soluble coating.

According to some embodiments of the invention, composition's buoyancy is affected by its form, surface features and density.

According to some embodiments of the invention, the composition is in a form selected from the group consisting of a capsule, ball, tablet, sachet, coated sachet, granules, wafer, sheet, powder, crystals, pellets, beads, pods, horseshoe, disk, holed disk, interlocked shapes, and a combination thereof.

According to some embodiments of the invention, the surface features of the composition comprise folds, indentations, channels, cut-outs, bubbles, hollows, air traps, rough surface, non-spherical shape, non-flat surface or a combination thereof.

According to some embodiments of the invention, the composition is configured to have a low density by adjusting a thickness of the composition, and adding air pockets, folds, indentations, channels, cut-outs, bubbles, or hollows.

According to some embodiments of the invention, the composition is packaged in single or multiple portions.

According to some embodiments of the invention, an amount of the composition for a single use is in a range between about 20 g to about 150 g.

According to some embodiments of the invention, maximum dimension of the composition is equal to or not more than 6 cm.

According to some embodiments of the invention, the composition is biodegradable.

According to an aspect of some embodiments of the invention, there is provided a method for reducing or preventing splashing on use of a toilet including: dropping the composition including 1/10-30% wt/wt of one or more surfactants, 5-40% wt/wt of one or more carbon dioxide evolving compounds and 5-40% wt/wt of one or more acids into the toilet; reacting the composition with water from the toilet to generate a foam; preventing splashing of the water from the toilet upon excretion the preventing due to the presence of the foam on a surface of the water; and flushing away the foam and excrement.

According to some embodiments of the invention, the composition releases an additional agent on reacting with water.

According to some embodiments of the invention, the dissolving or disintegrating of the foam releases an additional agent.

According to some embodiments of the invention, the foam generated on reacting covers at least part of a surface of the water in a toilet bowl.

According to some embodiments of the invention, the foam generated on reacting is generated within about 1 s to about 15 s.

According to some embodiments of the invention, a time to ¾ of maximum foam volume is less than 1 min.

According to some embodiments of the invention, the method further includes: forming a foam layer between 1 cm to about 10 cm thick.

According to some embodiments of the invention, the method further includes disintegrating half the foam in less than 12 mins.

According to some embodiments of the invention, the reacting of at least half of the compound is less than 15 mins.

According to some embodiments of the invention, the splashing is prevented or reduced by slowing or catching fecal matter.

According to some embodiments of the invention, the composition changes a characteristic of the water.

According to some embodiments of the invention, the method further includes dispensing of the composition from a dispenser.

### DESCRIPTION OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the Figures:

FIG. 1 is a flow diagram describing the use of the composition in accordance with an embodiment of the current invention;

FIG. 2 is a flow diagram describing the use of the composition in accordance with an embodiment of the current invention;

FIG. 3 is a flow diagram describing the use of the composition in accordance with an embodiment of the current invention;

FIG. 4 is a flow diagram describing the use of the composition in accordance with an embodiment of the current invention;

FIG. 5 is a schematic illustration of various exemplary configurations of the composition in accordance with an embodiment of the current invention;

FIGS. 6A and 6B are schematic illustrations of an exemplary configuration of the composition in accordance with an embodiment of the current invention;

FIG. 7: A schematic illustration of an exemplary overhead view of a composition expanding in a toilet bowl in accordance with an embodiment of the current invention;

FIG. 8 is a schematic illustration of a side view of a composition reacting with toilet water in accordance with an embodiment of the current invention;

FIG. 9 is a schematic illustration of a side view of a composition reacting with toilet water in accordance with an embodiment of the current invention; and

FIG. 10 is a flow diagram describing the use of the composition in accordance with an embodiment of the current invention.

### DESCRIPTION OF SPECIFIC EMBODIMENTS OF THE INVENTION

The present invention, in some embodiments thereof, relates to a composition for use in reducing splashing on excretion into a toilet, and optionally, the composition may be used to reduce noise and/or odor and/or to disinfect and/or to prevent accumulation of dirt on the walls of the toilet.

Overview

An aspect of some embodiments of the current invention relates to a composition for use in reducing splashing on excretion into a toilet is disclosed and described. Optionally, the composition may reduce noise and/or odor on excretion into a toilet. Optionally, the composition may disinfect a toilet after use. Optionally, the composition may remove and/or prevent accumulation of dirt on the inner walls of a toilet.

There is a need for an easily portable composition which prevents splashing for use in the home and in particular in public restrooms. Optionally, the composition may also reduce unpleasant noise while providing a pleasant scent, disinfection and cleaning of the toilet.

According to some embodiments, the composition may react with water, e.g., in the toilet bowl, to produce a foam. Optionally, the composition may include one or more carbon dioxide sources, one or more acids and a one or more surfactants. Optionally, the composition may include one or more additional excipient. Optionally, the composition may include one or more disintegrants.

According to some embodiments, the composition may be in the form of a tablet, capsule, wafer, sheet, powder, granules, crystals, etc. Optionally, the composition may float on or near the surface of the water-air interface. Optionally, the composition may rapidly dissolve and/or disintegrate in the water. Optionally, the composition may sink slowly. Optionally, the composition may be easily portable, e.g., easily transported and/or concealed in a pocket and/or a handbag and/or backpack and/or briefcase, etc. Unless otherwise stated, the word tablet is used herein in a general sense as any solid form of a chemical composition. For example, a tablet may be made up of compressed powder. Optionally a tablet may have any shape. Optionally, a tablet may have a homogenous and/or an heterogenous composition on different scales.

According to some embodiments, the foam generated on reaction with the toilet water may be a thick layer of carbon dioxide bubbles. Optionally, the composition may react rapidly with the water in the toilet bowl to produce a thick layer of foam. Optionally, the foam may allow matter to pass through it. Optionally, the foam may slow the passage of matter dropped onto it. Optionally, the foam may catch matter dropped onto it. Optionally the composition may change characteristics of the water (e.g., surface tension, density, viscosity). Optionally, the foam may dissipate after a period of time. Optionally, the foam may disintegrate after a period of time. Optionally, the foam and the waste may be flushed away. Optionally, the composition and/or the foam may not produce a blockage in the drain and/or drainage system. Optionally, the composition and/or the foam may be biodegradable.

According to some embodiments, the composition may include a pleasant scent which may be released on use. Optionally, the composition may be used to remove and/or prevent accumulation of dirt on the walls of the toilet. Optionally the composition may contain a disinfectant. For example, the composition may be dropped into a toilet, foam is generated upon contact with water, the foam reduces noise, splashing and/or unpleasant odors produced upon excretion into the toilet.

In some embodiments, the composition attempts to provide a solution to a widespread, but sometimes unspoken about problem, that might affect many people. Preventing the splashing of the water in the toilet on the falling may cause people discomfort, disgust and/or disease when this water splashes onto them or near to them.

In some embodiments, the splash effect can occur as a result of an object, like a rock for example, falling down into the water, the pressure of the object pushing into the water pushing the water around it in the upward position, resulting in a splash upwards. Some embodiments of the invention attempt to change how the pressure is applied to the water from the falling object e.g., excrement and/or other substances. This problem may be a worldwide problem, which may not have a widely used industrial solution, in the public bathrooms of North America, Europe, and other places. In parts of North America, the problem can be even more noticed because water level in the toilet can reach to ¾ of the height of the toilet bowl. In such a case, the splash can be relatively large, in this somewhat large amount of water, and can greatly discomfort the toilet user and/or a person sitting near the public and/or private toilet. Sometimes people, in attempt to prevent such a splash, before excreting, place in the toilet bowl substantial amounts of toilet paper and/or other substances which may result in clogging of the toilet system. Sometimes there can be multiple people in a short period of time attempting to put toilet paper or another substance to prevent the splash which can result in even more clogging. In addition, this practice can result in excessive use and/or waste of toilet paper and water, through multiple flushes to push the waste down the drain. Also, the splashing may result in noises which may further discomfort and/or embarrass the user in some public or private bathrooms.

In some embodiments, an additional problem that can occur: there could be bathrooms that require cleaning multiple times per day, and when not cleaned germs and/or potentially unhygienic and/or possibly harmful and/or contagious and/or unhealthy and/or unpleasant entities can build up in the toilet. As a result, of an unhygienic bathroom and/or toilet people may refrain from entering and/or may cause themselves damage by preventing themselves from excreting.

There may be a perceived need for a simple, light, not bulky, and inexpensive, product, which does not require extensive operation and extensive time, that may prevent unwanted actions, that will help maintain healthy and comfortable life. In some embodiments, the composition may advantageously preserve hygiene. In some embodiments, the composition may be suitable for any type of toilet. According to some embodiments, the composition may be used to sanitize the toilet and/or the water therein. According to some embodiments, the composition may require no extensive operation and/or does not require physical contact of the user with the toilet bowl and/or the water therein.

### Specific Embodiments

Before explaining at least one embodiment of the invention in detail, it is to be understood that the invention is not necessarily limited in its application to the details of construction and the arrangement of the components and/or methods set forth in the following description and/or illustrated in the drawings and/or the Examples. The invention is capable of other embodiments or of being practiced or carried out in various ways. Explanations of possible mechanisms of effect are not intended to limit the invention to a particular theoretical framework.

Some embodiments relate to a composition for use in reducing splashing on excretion into a toilet, and optionally, the composition may be used to reduce noise and/or odor and/or to disinfect and/or to prevent accumulation of dirt on the walls of the toilet.

According to some embodiments, the composition may float on and/or near the surface of the water-air interface. Optionally, the composition may rapidly dissolve and/or disintegrate in the water. Optionally, the composition may sink slowly. Optionally, the composition may be easily portable, e.g., easily transported and/or concealed in a pocket and/or a handbag and/or backpack and/or briefcase, etc.

According to some embodiments, the composition may react with water, e.g., in the toilet bowl, to produce a foam. Optionally, the composition may include one or more carbon dioxide sources, one or more acids and a one or more surfactants. Optionally, the composition may include one or more additional excipient. Optionally, the composition may include one or more disintegrants. In an embodiment, the foam may be continuously produced until all the material in the composition is used up, and then the foam may dissolve in the water and/or disintegrate over time.

According to some embodiments, the foam may allow matter to pass through it. Optionally, the foam may slow the passage of matter dropped onto it. Optionally, the foam may catch matter dropped onto it. Optionally the composition may change characteristics of the water (e.g., surface tension, density, viscosity). Optionally, the foam may dissipate after a period of time. Optionally, the foam may disintegrate after a period of time. Optionally, the foam and the waste may be flushed away. Optionally, the composition and/or the foam may not produce a blockage in the drain and/or drainage system. Optionally, the composition and/or the foam may be biodegradable. Optionally, the foam may be flushed away without clogging the toilet.

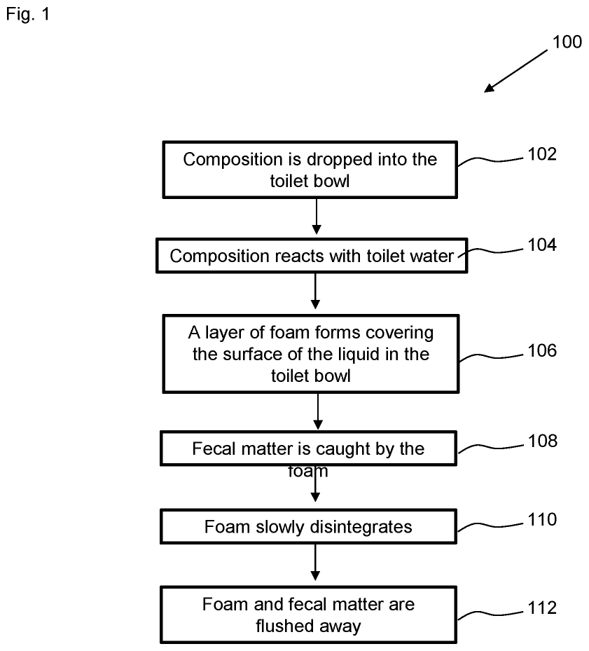

According to some embodiments, the foam may stop the fall of fecal matter into the toilet (e.g., FIG. 1). Optionally, the foam containing the fecal matter may then be flushed away. Optionally, as the foam disintegrates, it may gently deposit the fecal matter into the toilet. For example, the foam may catch the fecal matter thereby preventing splashing and noise.

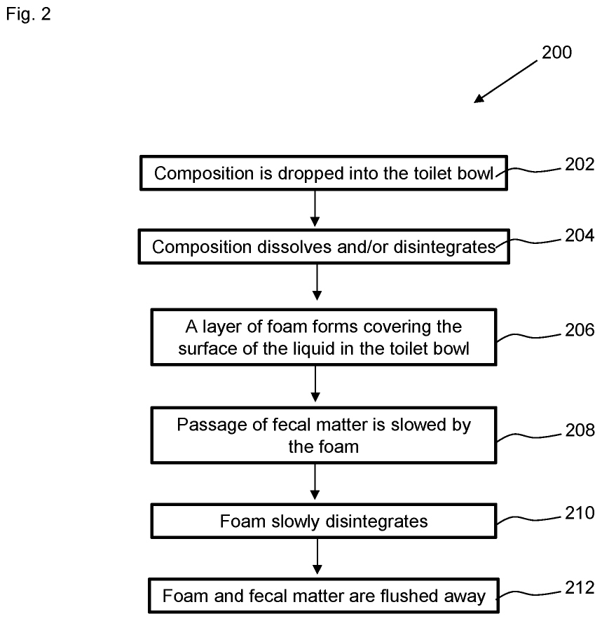

According to some embodiments, the foam may slow the passage of fecal matter into the toilet (e.g., FIG. 2). Optionally, the reduction in the velocity of the fecal matter may reduce splashing and noise.

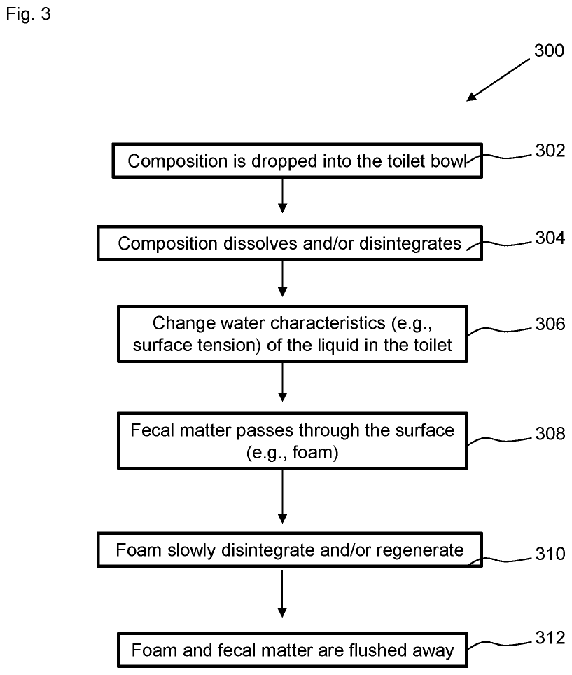

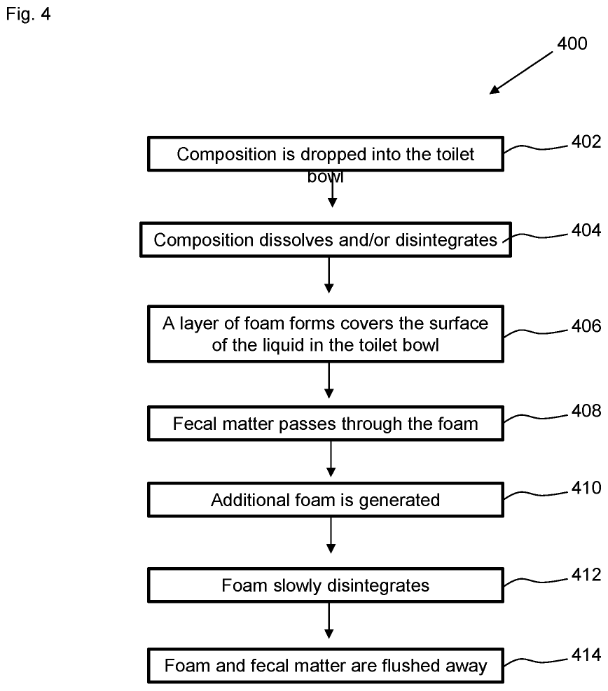

According to some embodiments, the composition may change the characteristics of the water through which fecal matter is to pass through (e.g., FIG. 3). For example, material of the composition may reduce the surface tension of the water. For example, the fecal matter may pass through a layer of foam. Optionally, the foam may continue to be generated to cover any gaps produced by passage of the fecal matter (e.g., FIG. 4) ready for additional activity. For example, the foam may prevent splashing and reduce noise.

In an embodiment, the composition may comprise of:
0.1-30% wt/wt one or more surfactants;
5-40% wt/wt one or more carbon dioxide evolving compounds; and
5-40% wt/wt one or more acids.

In an embodiment, one or more additional excipients may be added to the composition, for example, an odorant, a deodorant, a thickener, a disintegrant, a moisture absorbing agent, a disinfectant, a cleaning agent, a foam promoting agent, a colorant, binder, antiseptic, etc. and/or combinations thereof. Optionally, one or more of the compounds in the composition may serve a dual purpose. In an embodiment, any of the compounds and/or excipients included in the composition may be in the form of a liquid, particle, nanoparticle, powder, granule, flake, pellet, and/or combinations thereof.

In an embodiment, the one or more surfactants may be selected from anionic surfactants, cationic surfactants, amphoteric surfactants, non-ionic surfactants, saponins, proteins, etc. and/or a combination thereof. Optionally, the surfactants may be natural (for example natural fatty acids, coconut oil, palm oil, etc. and/or derivatives, adducts and/or salts thereof) or synthetic (for example fatty alcohol sulfates, fatty alcohol sulfonates, alkylarylsulfonates, alkyl aryl sulfonates, sulphonated benzimidazole derivatives, etc. and/or derivatives, adducts, salts, and/or combinations thereof). Each possibility is a separate embodiment.

In an embodiment, the anionic surfactant may be selected from alkylnaphthalene sulfonates, fatty alcohol sulphates or sulphonates which may be unsubstituted or substituted salts, adducts and/or derivatives thereof. In an embodiment, the anionic surfactant may be an ammonium or sodium salt, for example ammonium lauryl sulfate, sodium laureth sulfate, sodium lauryl sarcosinate, sodium myreth sulfate, sodium pareth sulfate, sodium stearte, sodium lauryl sulfate, α-olefin sulfonate, ammonium laureth sulfate, sodium isopropyl naphthalene sulfonate, sodium polystyrene sulfonate, etc. salts, adducts, derivatives, and/or combinations thereof. Each possibility is a separate embodiment.

In an embodiment, the cationic surfactant may be selected from quaternary ammonium salts in the form of halides, halogenated lower alkyl, benzyl, hydroxy-lower alkyl radicals, alkylsulfates which may be substituted or unsubstituted by halogens, alkyl group, and or aryl groups, and/or salts, adducts, derivatives, and/or combinations thereof, for example, ammonium chloride, ammonium hydroxide. Each possibility is a separate embodiment.

In an embodiment, the amphoteric surfactant may be selected from aminocarboxylic acids and aminosulphonic acids, alkyl and alkylamido betaines, higher fatty alkanolamides, and salts, adducts, derivatives, and/or combinations thereof. Each possibility is a separate embodiment.

In an embodiment, the non-ionic surfactant may be selected from polyalkylglycol ether or ester derivatives of aliphatic, cycloaliphatic or aryl alcohols, saturated or unsaturated fatty acids and alkylphenols, water-soluble glycol adducts of polyethylene oxide, fatty acid esters of polyoxyethylene sorbitan and/or salts, adducts, derivatives, and/or combinations thereof. Each possibility is a separate embodiment.

In an embodiment, the carbon dioxide evolving compound may be a carbonate. In an embodiment, the carbonate may be selected from ammonium carbonate, sodium carbonate, potassium carbonate, ammonium bicarbonate, sodium bicarbonate, potassium bicarbonate, sodium percarbonate, and/or other water-soluble carbonate and bicarbonate salts, adducts, derivatives, and/or combinations thereof. Each possibility is a separate embodiment. In an embodiment, more than one carbon dioxide evolving compound may be used.

In an embodiment, the acid may be organic or inorganic. In an embodiment, the acid may be selected from citric acid, tartaric acid, oleic acid, stearic acid, palmitic acid, glucuronic acid, α-hydroxybutyric acid, α-hydroxyisobutyric acid, mandelic acid, pyruvic acid, malic acid, citramalic acid, mucic acid, glycolic acid, hydroxyglutaric acid, lactic acid, catechol acid, oxalic acid, succinic acid, tartaric acid, salicyclic acid, adipic acid, ascorbic acid, acetic acid, boric acid, hydrochloric acid, hydrobromic acid, sulfuric acid, sulphamic acid, nitric acid and/or other water-soluble acids and/or combinations thereof. Each possibility is a separate embodiment. In an embodiment, more than one acid may be used.

In an embodiment, the composition may comprise a disintegrant. In an embodiment, the disintegrant may be selected from starch, partially pregelatinized starch, chitosan, mucilage, croscarmellose sodium, crospovidone, sodium starch glycolate, microcrystalline cellulose, and other commonly used disintegrants, and/or combinations thereof. Each possibility is a separate embodiment. In an embodiment, more than one disintegrant may be used.

In an embodiment, the composition may comprise a binder. In an embodiment, the binder may be selected from starch, pregelatinized starch, Sodium alginate, gelatin, polyvinyl pyrrolidone (PVP), methylcellulose, hydroxy propyl methyl cellulose (HPMC), polymethacrylates, sodium carboxy methyl cellulose, polyethylene glycol (PEG), methylcellulose, cellulose derivatives, polyvinylpyrrolidone, microcrystalline cellulose, and other commonly used binders, and/or combinations thereof. Each possibility is a separate embodiment. In an embodiment, more than one binder may be used.

In an embodiment, the composition may comprise a moisture absorbing agent. Alternatively, and/or additionally, the composition may comprise a moisture absorbing coating and/or layer comprising one or more moisture absorbing agents. In an embodiment, the moisture absorbing agent may be selected from aluminum oxide, calcium chloride, calcium sulfate, activated carbon, zeolites, silica gel, other common desiccants, and/or combinations thereof. Each possibility is a separate embodiment. Optionally, more than one moisture absorbing agent may be used.

In an embodiment, the composition may comprise an odorant and/or a deodorant. In an embodiment, the composition may release a pleasant odor on contact with water. Alternatively, and/or additionally, the foam may release a pleasant odor on formation and/or disintegration. Optionally, the odorant may be a perfume or fragrance. Optionally, the odorant may be natural (e.g., an essential oil or derivative thereof) or synthetic. Optionally, the deodorant may be a methane gas or hydrogen sulfide neutralizer.

In an embodiment, the composition may comprise a disinfectant. In an embodiment, the composition may release a disinfectant on contact with water. Alternatively, and/or additionally, the foam may release a disinfectant on formation and/or disintegration. Optionally, the disinfectant may be organic or inorganic. Optionally, the disinfectant may be selected from peroxides, percarbonates, peracids, chlorine compounds, phenolics, quaternary ammonium compounds, other common household disinfectants, and/or combinations thereof. Each possibility is a separate embodiment. Optionally, more than one disinfectant may be used.

In an embodiment, the composition may comprise a cleaning agent. In an embodiment, the composition may release a cleaning agent on contact with water. Alternatively, and/or additionally, the foam may release a cleaning agent on formation and/or disintegration. For example, the foam may clean the toilet and/or remove and/or prevent accumulation of fecal matter, urine and/or dirt on the walls and/or base of the toilet.

In an embodiment, the composition may comprise a foam promoting agent. Optionally, the foam promoting agent may be a buffer, a frothing agent, a foam stabilizer, a thickening agent, a foam booster and/or mixtures thereof. Each possibility is a separate embodiment. Optionally, more than one foam promoting agent may be used.

In an embodiment, the composition may be a capsule, ball, tablet, sachet, coated sachet, granules, wafer, sheet, powder, granules, crystals, pellets, beads, pods and/or a combination thereof. Each possibility is a separate embodiment. Optionally, a tablet may be circular, oval, horseshoe shaped, donut shaped, disk shaped, etc. Optionally, the tablet may be far from spherical (e.g., flat, horseshoe, thin [e.g., disk], rough, non-flat surface, etc.). Each possibility is a separate embodiment. Optionally, the composition may have a large surface area. Optionally, the composition may be configured to provide a large surface area. Optionally, the composition may include folds, indentations, channels, cut-outs, bubbles, hollows, etc. Each possibility is a separate embodiment. Optionally, the composition may be thin and/or light. Optionally, the composition may be light weight. Optionally, the composition may be configured to be light weight. Optionally, the composition may be configured to float. Optionally, the composition may be configured not to sink. Optionally, the composition may be configured not to sink rapidly. Optionally, the composition may be configured to bob to the surface. Optionally, the composition may be configured to prevent it from going down the drain prior to reaction with water.

In an embodiment, the composition may be packaged in an amount suitable for a single use. Optionally, the amount of the composition or a single use may be in the range between about 10 g to about 250 g, between about 20 g to about 200 g, between about 30 g to about 150 g, between about 40 g to about 100 g, or between about 50 g to about 80 g. Each possibility is a separate embodiment. Optionally, the composition may be packaged as a single portion. Optionally, the composition may be packaged as two or more portions. Optionally, the composition may be packaged by interlocking two or more portions.

In an embodiment, the composition may be used in the home and/or in public restrooms. Optionally, the composition may be provided in a dispenser. Optionally, the dispenser may be attached to a wall near the toilet. Optionally, the dispenser may be attached directly to the toilet. Optionally, activating the dispenser (e.g., by pressing on a handle, knob, button, etc.) a standard quantity (e.g., a single tablet, capsule, sachet, etc. or a pre-determined amount of powder, granules, liquid, etc.) may be dispensed into the toilet for immediate use. Optionally, a user may purchase an individual, single use composition.

In an embodiment the composition may be coated and/or have an outer layer to prevent absorption of moisture from the air and/or fingers of the user. Optionally, the outer layer may be soluble in water. In an embodiment, the composition may be compressed. Optionally, compression and/or coating may prevent crumbling, cracking and/or sticking.

In an embodiment, the composition may be equal to or not more than 7 cm, not more than 6.5 cm, not more than 6 cm, not more than 5.5 cm, not more than 5 cm, not more than 4.5 cm, not more than 4 cm, not more than 3.5 cm, not more than 3 cm, not more than 2.5 cm, not more than 2 cm, not more than 1.5 cm, not more than 1 cm, not more than 0.5 cm, not more than 0.25 cm, not more than 0.1 cm, not more than 0.05 cm, not more than 0.005 cm, or not more than 0.0005 cm in length and/or diameter. Each possibility is a separate embodiment.

In an embodiment, the composition volume may be equal to or less than 2,400 ml, less than 2,200 ml, less than 2,000 ml, less than 1,800 ml, less than 1,600 ml, less than 1,400 ml, less than 1,200 ml, less than 1,000 ml, less than 800 ml, less than 600 ml, less than 400 ml, less than 200 ml, or less than 100 ml. Each possibility is a separate embodiment.

In an embodiment, the composition may disintegrate slowly. In an embodiment, the composition may have a mean disintegration time of less than 15 mins, less than 12 mins, less than 10 mins, less than 8 mins, less than 6 mins, less than 5 mins, less than 3 mins, less than 2 mins, less than 1 min, less than half a min, less than 15 s, less than 10 s, or less than 5 s. Each possibility is a separate embodiment.

In an embodiment, the composition may dissolve slowly. In an embodiment, the composition may have a mean dissolution time of less than 15 mins, less than 12 mins, less than 10 mins, less than 8 mins, less than 6 mins, less than 5 mins, less than 3 mins, less than 2 mins, less than 1 min, less than half a minute, less than 15 s, less than 10 s, or less than 5 s. Each possibility is a separate embodiment.

According to some embodiments, the foam generated on reaction of the composition with the toilet water may cover part and/or all of the liquid surface and/or the walls in a toilet bowl. Optionally, the thickness of the foam layer may be between about 0.25 cm to about 15 cm, between about 0.5 cm to about 12.5 cm, between about 1 cm to about 10 cm, or between about 2 cm to about 7.5 cm. Each possibility is a separate embodiment.

According to some embodiments, the foam generated on reaction of the composition with the toilet water may be a thick layer of bubbles. Optionally, the composition may react rapidly with the water in the toilet bowl to produce a thick layer of foam. Optionally, the foam may be generated within about 1 s to about 120 s, within about 1 s to about 90 s, within about 1 s to about 60 s, within about 1 s to about 30 s, within about 1 s to about 20 s, within about 1 s to about 15 s or within about 1 s to about 10 s. Each possibility is a separate embodiment. In some embodiments, the foam continues to be generated at least at a replenishment rate (that rate of generation of the foam of greater that a rate of disintegration of the from and/or at a rate high enough to preserve the thickness and/or density of the foam later on the water surface) for between 1 to 5 minutes and/or between 5 to 10 minutes and/or between 10 to 20 minutes.

In an embodiment, the foam may be formed rapidly. In an embodiment, the time to ¾ of maximum foam volume may be less than 5 s, 10 s, less than 20 s, less than 30 s, less than 40 s, less than 50 s, less than 1 min, less than 1.5 mins, less than 2 mins, less than 2.5 mins, less than 3 mins, less than 3.5 mins, less than 4 mins, less than 4.5 mins, or less than 5 mins. Each possibility is a separate embodiment.

In an embodiment, the disintegration time to ⅓ maximum foam volume may be more than 1/4 min, more than 1/2 min, more than 1 min, more than 1.5 mins, more than 2 mins, more than 2.5 mins, more than 3 mins, more than 3.5 mins, more than 4 mins, more than 4.5 mins, more than 5 mins, more than 5.5 mins, more than 6 mins, more than 6.5 mins, more than 7 mins, more than 7.5 mins, more than 8 mins, more than 8.5 mins, more than 9 mins, more than 9.5 mins, or more than 10 mins. Each possibility is a separate embodiment. For example, the noise and splash prevention effect can be produced over the duration of the activity.

While the invention has been described in its preferred form or embodiment with some degree of particularity, it is understood that this description has been given only by way of example and that numerous changes in the details of construction, fabrication, and use, including the combination and arrangement of parts, may be made without departing from the spirit and scope of the invention.

Some embodiments, the composition may solve some or all the problems listed above, under certain conditions. For example, the composition in the form of a tablet may be thrown into the water of the toilet. Optionally, contact with the water, under the right conditions, may cause the tablet to expand rapidly, for example within seconds. Optionally, the tablet may expand like a flower, at the air-water interface, or causing it to touch and/or grasp the walls and/or of the bottom of the toilet bowl.

In some embodiments, the composition may have one or more advantages. For example, prevention of splashing water onto the person sitting on the toilet.

In some embodiments, the composition may include a sheet of paper which may be strong enough to not dissolve immediately in the water of the toilet in order to provide the desired effect, but may also disintegrate in water and therefore may not cause to problems in the pipes of the toilet. In some embodiments. the composition may not contain oils that are used in various wipes to make them softer to the skin, which may cause clogging of the pipes.

In some embodiments, on contact with the water the composition expands to a sheet and/or curls upward, for example at the outer edges. The sheet may optionally continue to expand until it reaches the walls of the toilet bowl. The upward curled edges of the sheet may optionally protect the walls of the toilet from becoming soiled. Optionally, there may be different sized compositions and/or tablets. For example, a larger composition may be configured to spread over surfaces of larger toilets. Optionally, the sheet contacts the inner walls of the toilet. In some embodiments, as the edges of the composition reach the walls of the toilet, they may continue to expand upward along the walls the toilet. For example, this may protect the walls of the toilet from becoming soiled. Optionally, there may be different sized composition and/or tablets. For example, a larger composition may be configured to spread over surfaces of larger toilets.

In some embodiments, the composition may be to remove foul odors and/or provide good smells. For example, on contact of the composition with the toilet water it may release fragrant substances, that provide a good scent. There are many compositions for the purpose of providing good scent. Among them are: air fresher, to provide a good scent after the use of the bathroom, or devices which are built onto or hanging from the toilet which drop liquid and spread a good scent at the time of the flushing the water. In some embodiments, the composition may provide a good scent even before the flushing of the toilet, and/or even before the act of excreting.

In some embodiments, the composition may prevent the buildup of foul odors over time from excrement and/or other filth. Generally, most of the excrement and filth in the toilet centralizes in the bottom of the toilet bowl, however according to some embodiments. the composition may reduce and/or prevent the buildup, allowing it to easily be entirely flushed away with the composition after use. Optionally, the composition may shield the bottom of the toilet from sticking excrement and/or the material of the composition which the excrement is on, itself gets easily flushed and/or draws the excrement down the drain. In some embodiments, this may reduce the amount of constant cleaning required in some bathrooms and/or could reduce the buildup of germs. In some embodiments, the composition may contain antibacterial substances, as is known that excrement contains many germs. There are toilets which might have various wedges, nooks and/or crannies in the toilet bowl in which filth gets stuck a can build up and even be hard to clean. In some embodiments, the composition may prevent filth from getting caught in wedges, nooks and/or crannies in the toilet bowl. For example, the filth may be easily flushed away along with the composition after use. In addition, in some embodiments, the composition may dampen or remove the noise of excrement falling into the water. This is a problem which may yet not have an industrial solution which is widespread. In some embodiments, the composition may be small and/or compact enabling it to not take up much space. In some embodiments, the composition may be easily portable. In some embodiments, the composition may be packaged with illustrated pictures for children which may encourage children to also use the composition and even encourage them to potty train and/or move from a potty to use a full toilet.

List of some aspects of some advantages of composition according to some embodiments follow:
Splashing—may reduce and/or prevent the splash of water that spurts up from the water in the toilet upon entry of waste into the toilet, touching the body of the user sitting on the toilet or near to the toilet.
Noise—may reduce and/or prevent the sound that is caused by excrement falling into the water.
Smell—may reduce and/or prevent foul smells and spreads good scent while the toilet is in use and/or thereafter.
Antibacterial substance—may reduce and/or prevent the buildup of germs and bacteria on the bottom and/or walls of the toilet bowl.
Long term smells—may reduce and/or prevent the buildup of filth and reduces the need to constantly clean the toilet.

In some embodiments, the composition may be eco-friendly, e.g., it may be biodegradable, break up in water and/or may not cause clogging. In some embodiments, the composition may be sufficiently sturdy to withstand some time in water to fulfill its purpose and/or expands nearly instantaneously, (e.g., in seconds), when touched with the water of the toilet. In some embodiments, the composition may be compact, portable, possible to be in every bathroom as is toilet paper, both public and private bathrooms and/or encourages children to potty train.

Reference is now made to the figures.

FIG. 1 is a flow diagram describing the use of the composition in accordance with an embodiment of the current invention. For example, in the process 100, the composition may be dropped into the toilet bowl 102 where the composition may react with the toilet water 104, to form a layer of foam which may cover all or part or the surface of the liquid in the toilet bowl 106. The fecal matter may be caught by the foam 108, preventing splashing. The foam may slowly disintegrate 110, gently depositing the fecal matter into the base of the toilet or the fecal matter and any remaining foam are then flushed away 112, leaving the toilet bowl fresh and clean for the next user.

Optionally, the foam may be thick enough to catch the fecal matter. Optionally, the foam generated on reaction of the composition with the toilet water may be a thick layer of bubbles. Optionally, the thickness of the foam layer may be between about 0.25 cm to about 15 cm, between about 0.5 cm to about 12.5 cm, between about 1 cm to about 10 cm, or between about 2 cm to about 7.5 cm.

FIG. 2 is a flow diagram describing the use of the composition in accordance with an embodiment of the current invention. For example, in the process 200, the composition may be dropped into the toilet bowl 202 where the composition may react with the toilet water 204, to form a layer of foam which may cover all or part or the surface of the liquid in the toilet bowl 206. Fecal matter may be dropped into the toilet and passage of the fecal matter is slowed by the foam, preventing noise and splashing 208, the foam may then disintegrate 210, gently depositing the fecal matter into the base of the toilet or the fecal matter and any remaining foam are then flushed away 212, leaving the toilet bowl fresh and clean for the next user.

Optionally, the time to ¾ of maximum foam volume may be less than 5 s, 10 s, less than 20 s, less than 30 s, less than 40 s, less than 50 s, less than 1 min, less than 1.5 mins, less than 2 mins, less than 2.5 mins, less than 3 mins, less than 3.5 mins, less than 4 mins, less than 4.5 mins, or less than 5 mins.

FIG. 3 is a flow diagram describing the use of the composition in accordance with an embodiment of the current invention. For example, in the process 300, the composition may be dropped into the toilet bowl 302 where the composition may react with the toilet water 304, to form a layer of foam which may cover all or part or the surface of the liquid in the toilet bowl 306. Fecal matter is dropped into the toilet and passes through the foam, reducing noise and preventing splashing 308, the foam may then disintegrate 310, gently depositing the fecal matter into the base of the toilet or the fecal matter and any remaining foam are then flushed away 312, leaving the toilet bowl fresh and clean for the next user.

Optionally, the foam may absorb and/or prevent the water from an upward splash from rising above the upper surface of the foam.

FIG. 4 is a flow diagram describing the use of the composition in accordance with an embodiment of the current invention. For example, in the process 400, the composition may be dropped into the toilet bowl 402 where the composition may react with the toilet water 204 and/or react with toilt, to form a layer of foam which may cover all or part or the surface of the liquid in the toilet bowl 406. Fecal matter may be dropped into the toilet and passage of the fecal matter is slowed by the foam, preventing noise and splashing 408. Additional foam may be generated to fill in any gaps thereby formed, for continued activity 410. The foam may then disintegrate 412, gently depositing the fecal matter into the base of the toilet or the fecal matter and any remaining foam are then flushed away 414, leaving the toilet bowl fresh and clean for the next user.

Optionally, the composition may comprise multiple portions of the composition. Optionally, some of the portions of the composition may be configured to disintegrate and/or dissolve and/or react slower than others. Optionally, this may ensure continuous production of foam over several minutes. Optionally, this may provide waves of foam which may replace areas of foam as it is depleted.

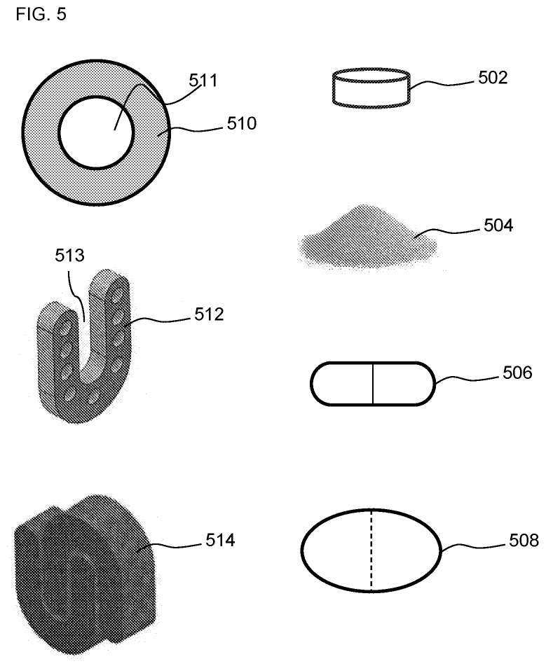

FIG. 5 is a schematic illustration of various exemplary configurations of the composition in accordance with an embodiment of the current invention. For example, the composition may be in the form of a capsule 506, ball, tablet 502, sachet, coated sachet, granules, wafer, sheet 508, powder 504, granules, crystals, pellets, beads, pods, disk, donut (disk with a hole) 510, horseshoe shaped 512, interlocking shapes 514, and/or a combination thereof. In some embodiments, the composition may be encased in a wrapper. Optionally, the composition may not include an individual wrapper. Optionally, the composition may be packaged as a single portion. Optionally, the composition may be packaged as two or more portions. Optionally, the composition may be packaged by interlocking two or more portions. Optionally, the portions may have surface features, such as indentations, hollows, etc. For example, hollows and/or smaller portions and/or different shaped portions may increase surface area and/or reaction rate and/or gas retention and/or buoyancy of the portions. Optionally, a single portion may be used in a single use, e.g., 2 tablets. Optionally, multiple portions may be used in a single use. Optionally, multiple portions may be used in multiple uses. Optionally, the composition may be in the form of multiple small tablets. Optionally, the composition may be in the form of large tablet. Optionally, the composition may be in the form of a powder, crystals and/or granules. Optionally, the powder may sink below the surface of the water. Optionally, the powder may be rapidly wet. Optionally, only a small amount of the powder may float on the surface. Optionally, only a small amount of the composition may remain unreacted.

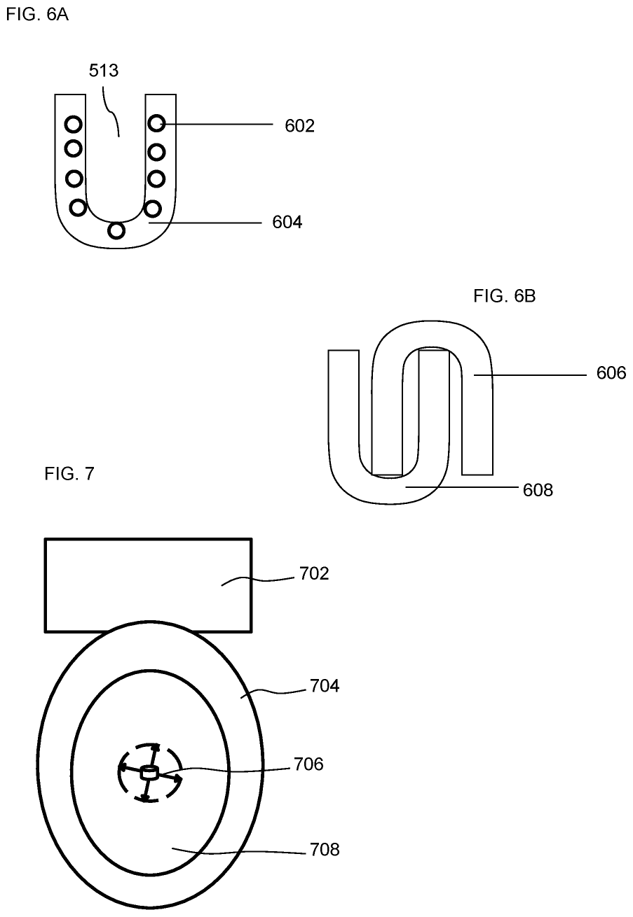

FIGS. 6a and 6b are a schematic illustration of exemplary configurations of the composition in accordance with an embodiment of the current invention.

According to some embodiments, the composition should not sink into the toilet trap and/or drain. Optionally, the buoyancy of the composition may be affected by its shape and/or surface features.

According to some embodiments, the composition may be in the form of a capsule, ball, tablet, sachet, coated sachet, granules, wafer, sheet, powder, granules, crystals, pellets, beads, pods, disk, donut (disk with a hole), horseshoe shaped 604, interlocking shapes 606 and 608, and/or a combination thereof. Optionally, the composition may be configured to provide a large surface area. Optionally, the composition may include surface features such as folds, indentations, channels, cut-outs, bubbles, hollows, etc. Optionally, the composition may have a large surface area. Optionally, the composition may be configured to provide a large surface area. Optionally, the composition may include folds, indentations 602, channels, cut-outs 511, 513, bubbles, hollows, etc. Optionally, the surface features may increase the surface area. Optionally, the surface features (e.g., indentations) may trap air. Additionally or alternatively, the tablets may include internal voids and/or pockets (e.g., to increase buoyancy). Optionally, the large surface area may increase the speed reaction, disintegration and/or carbon dioxide production, thereby preventing sinking.

According to some embodiments, the composition may have a low density. Optionally, the composition may be thin and/or light. Optionally, the composition may be light weight. Optionally, the composition may be configured to be light weight. Optionally, the composition may include air pockets, surface features configured to trap air, rough surface, non-spherical shape (e.g., having protrusions and/or recesses and/or a non-convex shape and/or an irregular shape and/or a large ratio between dimensions [e.g., the ratio between the longest and shortest dimensions may be between 3 to 5 and/or 5 to 10 and/or between 10 to 50 and/or between 50 to 100 and), non-flat surface, etc. Optionally, the composition may be configured to float. Optionally, the composition may be configured not to sink. Optionally, the composition may be configured not to sink rapidly. Optionally, the composition may be configured to bob to the surface. Optionally, the composition may be configured to prevent it from going down the drain prior to reaction with water.

FIG. 7 is a schematic illustration of an exemplary overhead view of a composition expanding in a toilet bowl in accordance with an embodiment of the current invention. For example, the composition 706 may be dropped into the toilet bowl 708 (the inner oval is the internal walls of the toilet). Optionally, the tablet opens to a sheet or foam that may float on the surface of the water in the toilet and/or expand towards the walls of the toilet bowl. Optionally, when a user sits on the toilet seat 704, they may not be splashed by any backsplash due to the use of the composition, which may be easily flushed away by water from the toilet cistern 702 after use. Optionally, the foam may spread out from the compositions point of entry into the toilet bowl. Optionally, the foam may cover all or part of the surface of the water in the toilet bowl.

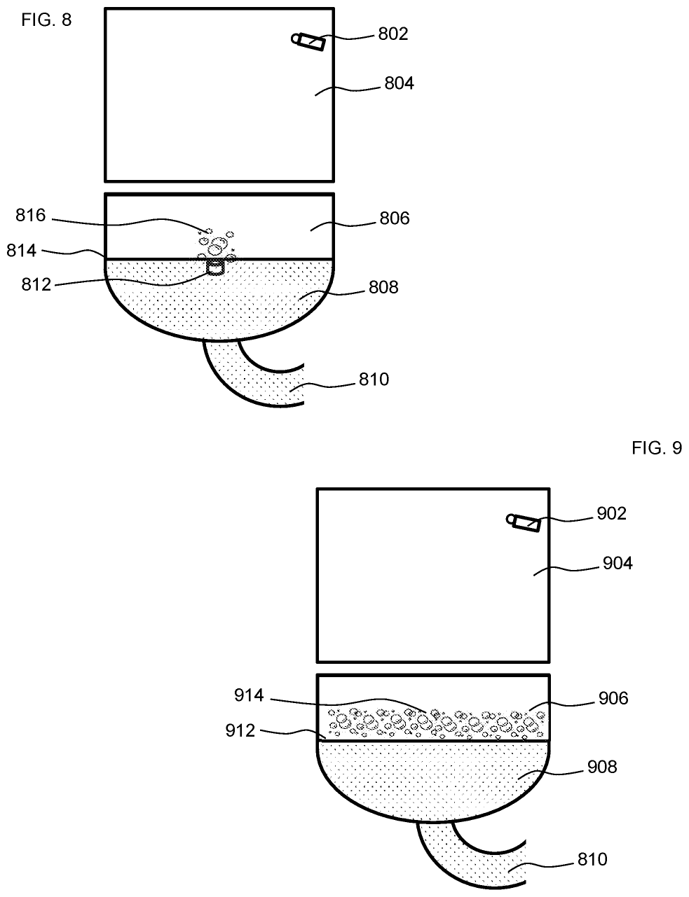

FIG. 8 is a schematic illustration of a side view of a composition reacting with toilet water in accordance with an embodiment of the current invention. For example, the composition 812 may be dropped into the toilet bowl 806. The composition 812 may react with the water in 808 in the toilet bowl 806, e.g., to evolve a foam 816 at the air-water interface 814. The composition may disintegrate after a certain period of time and may be easily flushed away by water from the toilet cistern 804 on depression of the handle 802, after use, without clogging the drain 810. Optionally, the buoyancy of the composition may prevent it from sinking and/or passing into the trap and/or drain prior to and/or during reaction with the water in the toilet. Optionally, the compositions buoyancy may be affected by the compositions shape, size, surface features and/or density. Optionally, the composition may be equal to or not more than 7 cm, not more than 6.5 cm, not more than 6 cm, not more than 5.5 cm, not more than 5 cm, not more than 4.5 cm, not more than 4 cm, not more than 3.5 cm, not more than 3 cm, not more than 2.5 cm, not more than 2 cm, not more than 1.5 cm, not more than 1 cm, not more than 0.5 cm, not more than 0.25 cm, not more than 0.1 cm, not more than 0.05 cm, not more than 0.005 cm, or not more than 0.0005 cm in length and/or diameter.

FIG. 9 is a schematic illustration of a side view of a composition reacting with toilet water in accordance with an embodiment of the current invention. For example, the composition 912 may be dropped into the toilet bowl 906. The composition 912 may react with the water in 908 in the toilet bowl 906, e.g., to evolve a foam 916 at the air-water interface 914 which may cover all or part of the water surface. The composition may disintegrate after a certain period of time and may be easily flushed away by water from the toilet cistern 904 on depression of the handle 902, after use, without clogging the drain 910.

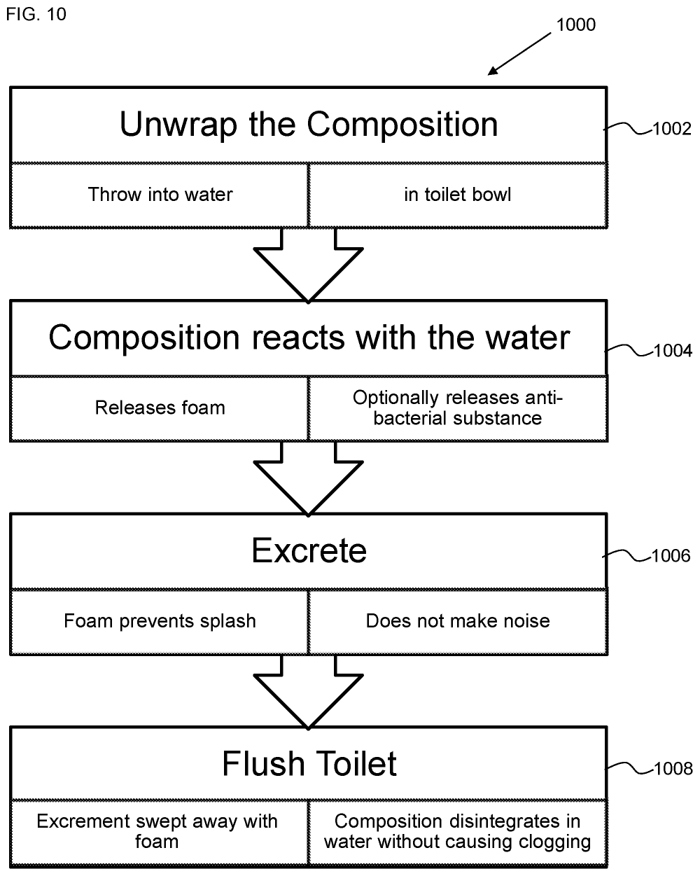

FIG. 10 is a flow diagram describing the use of the composition in accordance with an embodiment of the current invention. For example, in the process 1000, the composition is unwrapped 1002 and thrown into the water in a toilet bowl. The composition reacts with the water 1004, to release foam, and optionally an anti-bacterial and/or odor control substance. The user then excretes 1006 into the toilet bowl where the foam reduces and/or prevents splashing and/or noise. After use, the toilet is then flushed 1008, sweeping away the excrement and the foam, without causing clogging of the drain since the composition disintegrates in the water.

These embodiments are provided by way of example and are in no means intended to be limiting the scope of the invention.

### EXAMPLES

Reference is now made to the following examples, which together with the above descriptions illustrate some embodiments of the invention in a non limiting fashion.

### Example 1: Powder

Weight of the powder was between 50-80 grams. Optional formulations may include some or all of the ingredients and/or other ingredients.

| | Wt % in | |
|---|---|---|
| Ingredient | Formula | Range |
| Sodium carbonate | 8.00% | 2-12% |
| Sodium Bicarbonate | 23.00% | 15-30% |
| Blue Ocean 111 fragrance | 1.50% | 0.5-3% |
| Aerosil 200 (fumed silica) | 1.00% | 0.5-2% |
| Microcrystalline cellulose | 12.00% | 8-15% |
| Ginasul 68 (alpha olefin sulfonate) | 4.20% | 2-8% |
| Sodium Percarbonate | 3.90% | 2-8% |
| Ascorbic Acid | 1.50% | 0.5-3% |
| Sulphamic Acid | 44.88% | 30-60% |
| Blueor 1515 (blue color) | 0.02% | 0.01-0.04% |
| Total | 100% | |

The composition reacted rapidly with the toilet water, i.e., 3-8 seconds.

The height of the foam was between 2-7 centimeters at least (depending on the structure of the toilet). The foam remained thick and stable foam from the moment addition to the toilet water for between 8-15 minutes

### Example 2: Granules

Weight of the granules was between 50-80 grams. Optional formulations may include some or all of the ingredients and/or other ingredients.

| Ingredient | Wt % in Formula | Range |
|---|---|---|
| Sodium carbonate | 8.00% | 2-12% |
| Sodium Bicarbonate | 23.00% | 15-30% |
| Blue Ocean 111 fragrance | 1.50% | 0.5-3% |
| Aerosil 200 (fumed silica) | 1.00% | 0.5-2% |
| Microcrystalline cellulose | 12.00% | 8-15% |
| Ginasul 68 (alpha olefin sulfonate) | 4.20% | 2-8% |
| Sodium Percarbonate | 3.90% | 2-8% |
| Ascorbic Acid | 1.50% | 0.5-3% |
| Sulphamic Acid | 44.88% | 30-60% |
| Blueor 1515 (blue color) | 0.02% | 0.01-0.04% |
| Total | 100% | |

The composition reacted rapidly with the toilet water, i.e., 3-8 seconds.

The height of the foam was between 2-7 centimeters at least (depending on the structure of the toilet). The foam remained thick and stable foam from the moment addition to the toilet water for between 8-15 minutes

### Example 3: Tablet

Weight of the tablet was between 50-80 grams.

| Ingredient | Wt % in Formula | Range |
|---|---|---|
| Sodium carbonate | 10.00% | 5-20% |
| Sodium Bicarbonate | 32.88% | 15-50% |
| Blue Ocean 111 fragrance | 1.50% | 0.5-3% |
| Polyvinyl Pyrrolidone 90 | 0.60% | 0.3-2% |
| Microcrystalline cellulose | 4.40% | 2-8% |
| Ginasul 68 (alpha olefin sulfonate) | 6.20% | 3-12% |
| Sodium Percarbonate | 3.90% | 2-8% |
| Ascorbic Acid | 5.50% | 3-10% |
| Sulphamic Acid | 35.00% | 20-60% |
| Blueor 1515 (blue color) | 0.02% | 0.01-0.04% |
| Total | 100% | |

The composition reacted rapidly with the toilet water, i.e., 3-8 seconds.

The height of the foam was between 2-7 centimeters at least (depending on the structure of the toilet). The foam remained thick and stable foam from the moment addition to the toilet water for between 8-15 minutes

### General

It is expected that during the life of a patent maturing from this application many relevant building technologies, artificial intelligence methodologies, computer user interfaces, image capture devices will be developed and the scope of the terms for design elements, analysis routines, user devices is intended to include all such new technologies a priori.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

As used herein the term “about” refers to ±10%

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination or as suitable in any other described embodiment of the invention. Certain features described in the context of various embodiments are not to be considered essential features of those embodiments, unless the embodiment is inoperative without those elements.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

What is claimed is:

1. A composition to reduce or prevent splashing on use of a toilet comprising:
1/10-30% wt/wt of one or more surfactants;
5-40% wt/wt of one or more carbon dioxide evolving compounds; and
5-40% wt/wt of one or more acids.

2. The composition of claim 1, further comprising a disintegrant.

3. The composition of claim 1, further comprising one or more additional excipients selected from the group consisting of an odorant, a deodorant, a thickener, a disintegrant, a binder, a moisture absorbing agent, a disinfectant, a cleaning agent, a foam promoting agent, a colorant, and combinations thereof.

4. The composition of claim 3, wherein at least one the additional excipients in the composition serves a dual purpose.

5. The composition of claim 1, wherein the one or more surfactant includes at least one of anionic surfactants, cationic surfactants, amphoteric surfactants, non-ionic surfactants, saponins, proteins, or a combination thereof.

6. The composition of claim 5, wherein the one or more surfactants includes a natural or synthetic compound.

7. The composition of claim 1, wherein the one or more surfactants includes at least one compound selected from the group consisting of alkylnaphthalene sulfonates, fatty alcohol sulphates or sulphonates which may be unsubstituted or substituted salts, adducts and derivatives thereof.

8. The composition of claim 1, wherein the one or more surfactants includes at least one compound selected from the group selected from the group consisting of an ammonium salt, a sodium salt, ammonium lauryl sulfate, sodium laureth sulfate, sodium lauryl sarcosinate, sodium myreth sulfate, sodium pareth sulfate, sodium stearte, sodium lauryl sulfate, α-olefin sulfonate, ammonium laureth sulfate, sodium isopropyl naphthalene sulfonate, sodium polystyrene sulfonate, and salts, adducts, derivatives, and combinations thereof.

9. The composition of claim 1, wherein the one or more surfactants includes at least one compound selected from the group consisting of quaternary ammonium salts in the form of halides, halogenated lower alkyl or benzyl or hydroxy-lower alkyl radicals, alkylsulfates which may be substituted or unsubstituted by halogens, alkyl group, and or aryl groups, and/or salts, adducts adducts, derivatives, and combinations thereof.

10. The composition of claim 1, wherein the one or more surfactants includes at least one compound selected from the group consisting of aminocarboxylic acids and aminosulphonic acids, alkyl and alkylamido betaines, higher fatty alkanolamides, salts, adducts adducts, derivatives and combinations thereof.

11. The composition of claim 1, wherein t the one or more surfactants includes at least one compound selected from the group consisting of polyalkylglycol ether or ester derivatives of aliphatic, cycloaliphatic or aryl alcohols, saturated or unsaturated fatty acids and alkylphenols, water-soluble glycol adducts of polyethylene oxide, fatty acid esters of polyoxyethylene sorbitan, salts, adducts, derivatives, and combinations thereof.

12. The composition of claim 1, wherein the one or more carbon dioxide evolving compound includes a carbonate.

13. The composition of claim 1, wherein the one or more carbon dioxide evolving compound is selected from ammonium carbonate, sodium carbonate, potassium carbonate, ammonium bicarbonate, sodium bicarbonate, potassium bicarbonate, adducts, derivatives or combinations thereof.

14. The composition of claim 1, wherein the at least one acid is organic or inorganic.

15. The composition of claim 14, wherein the at least one the acid is selected from the group consisting of citric acid, tartaric acid, oleic acid, stearic acid, palmitic acid, glucuronic acid, α-hydroxybutyric acid, α-hydroxyisobutyric acid, mandelic acid, pyruvic acid, malic acid, citramalic acid, mucic acid, glycolic acid, hydroxyglutaric acid, lactic acid, catechol acid, oxalic acid, succinic acid, tartaric acid, salicyclic acid, adipic acid, ascorbic acid, acetic acid, boric acid, hydrochloric acid, hydrobromic acid, sulfuric acid, sulphamic acid, nitric acid, and any combination thereof.

16. The composition of claim 2, wherein the at least one disintegrant is selected from the group consisting of starch, partially pregelatinized starch, chitosan, mucilage, croscarmellose sodium, crospovidone, sodium starch glycolate, microcrystalline cellulose, and combinations thereof.

17. The composition of claim 3, wherein the additional excipient is a moisture absorbing agent.

18. The composition of claim 17, wherein the moisture absorbing agent is included in a coating.

19. The composition of claim 17, wherein the moisture absorbing agent is selected from the group consisting of aluminum oxide, calcium chloride, calcium sulfate, activated carbon, zeolites, silica, and a combination thereof.

20. The composition of claim 3, wherein the additional excipient is an odorant.

21. The composition of claim 20, wherein the odorant is a perfume or fragrance.

22. The composition of claim 3, wherein the additional excipient is a deodorant.

23. The composition of claim 22, wherein the deodorant is a methane gas or hydrogen sulfide neutralizer.

24. The composition of claim 3, wherein the additional excipient is a binder.

25. The composition of claim 24, wherein the binder is selected from the group consisting of starch, pregelatinized starch, Sodium alginate, gelatin, polyvinyl pyrrolidone (PVP), methylcellulose, hydroxy propyl methyl cellulose (HPMC), polymethacrylates, sodium carboxy methyl cellulose, polyethylene glycol (PEG), methylcellulose, cellulose derivatives, polyvinylpyrrolidone, microcrystalline cellulose, and combinations thereof.

26. The composition of claim 3, wherein the additional excipient is a disinfectant.

27. The composition of claim 26, wherein the disinfectant is selected from peroxides, percarbonates, peracids, chlorine compounds, phenolics, quaternary ammonium compounds, or combinations thereof.

28. The composition of claim 3, wherein the additional excipient is a cleaning agent.

29. The composition of claim 3, wherein the additional excipient is a foam promoting agent.

30. The composition of claim 29, wherein the foam promoting agent is a buffer, a frothing agent, a foam stabilizer, a thickening agent, a foam booster, or combinations thereof.

31. The composition of claim 1, further comprising a water soluble coating.

32. The composition of claim 1, wherein composition's buoyancy is affected by its form, surface features and density.

33. The composition of claim 32, wherein the composition is in a form selected from the group consisting of a capsule, ball, tablet, sachet, coated sachet, granules, wafer, sheet, powder, crystals, pellets, beads, pods, horseshoe, disk, holed disk, interlocked shapes, and a combination thereof.

34. The composition of claim 32, wherein the surface features of the composition comprise folds, indentations, channels, cut-outs, bubbles, hollows, air traps, rough surface, non-spherical shape, non-flat surface or a combination thereof.

35. The composition of claim 32, wherein the composition is configured to have a low density by adjusting a thickness of the composition, and adding air pockets, folds, indentations, channels, cut-outs, bubbles, or hollows.

36. The composition of claim 32, wherein the composition is packaged in single or multiple portions.

37. The composition of claim 1, wherein an amount of the composition for a single use is in a range between about 20 g to about 150 g.

38. The composition of claim 1, wherein maximum dimension of the composition is equal to or not more than 6 cm.

39. The composition of claim 1, wherein the composition is biodegradable.

40. A method for reducing or preventing splashing on use of a toilet comprising:
dropping the composition including
1/10-30% wt/wt of one or more surfactants,
5-40% wt/wt of one or more carbon dioxide evolving compounds and
5-40% wt/wt of one or more acids
into the toilet;
reacting the composition with water from the toilet to generate a foam;
preventing splashing of the water from the toilet upon excretion said preventing due to the presence of said foam on a surface of said water; and
flushing away the foam and excrement.

41. The method of claim 40, wherein the composition releases an additional agent on reacting with water.

42. The method of claim 40, wherein the dissolving or disintegrating of the foam releases an additional agent.

43. The method of claim 40, wherein the foam generated on reacting covers at least part of a surface of the water in a toilet bowl.

44. The method of claim 40, wherein the foam generated on reacting is generated within about 1 s to about 15 s.

45. The method of claim 40, wherein a time to ¾ of maximum foam volume is less than 1 min.

46. The method of claim 40, further comprising: forming a foam layer between 1 cm to about 10 cm thick.

47. The method of claim 40, further comprising disintegrating half the foam in less than 12 mins.

48. The method of claim 40, wherein the reacting of at least half of the compound is less than 15 mins.

49. The method of claim 40, wherein the splashing is prevented or reduced by slowing or catching fecal matter.

50. The method of claim 40, wherein the composition changes a characteristic of the water.

51. The method of claim 40, further comprising dispensing of the composition from a dispenser.
